(19) United States
(12) Patent Application Publication
(10) Pub. No.: US 2009/0281183 A1
(43) Pub. Date: Nov. 12, 2009

(54) L-CARNITINE CALCIUM FUMARATE, PREPARATION METHOD AND APPLICATION FOR THE SAME

(75) Inventors: Xueliang Yuan, Liaoning (CN); Guoqing Mei, Liaoning (CN); Duqing Qing, Liaoning (CN); Weiwei Zhang, Liaoning (CN); Ling Chen, Liaoning (CN)

Correspondence Address: DAVID AND RAYMOND PATENT FIRM, 108 N. YNEZ AVE., SUITE 128, MONTEREY PARK, CA, 91754, US

(73) Assignee: SHENYANG KONCEPNUTRA CORPORATION, LIMITED, Shenyang (CN)

(21) Appl. No.: 12/310,192

(22) PCT Filed: Oct. 19, 2007
(86) PCT No.: PCT/CN2007/003001

§ 371 Date: Feb. 13, 2009

(30) Foreign Application Priority Data
Dec. 29, 2006 (CN) 200610135198.X
Aug. 28, 2007 (CN) 200710148636.0

(51) Int. Cl. A61K 31/205; C07C 57/15; A61P 9/10

(52) U.S. Cl. 514/556; 562/598

(57) ABSTRACT

L-carnitine calcium fumarate and its preparation and applications are disclosed in the present invention. Having good water solubility as well as better and stronger nutritious and treating functions than the corresponding inner salts, the L-carnitine calcium fumarate as proposed having the advantages of being non-hygroscopic, stable in chemical property, and suitable for oral administration. A preparing method is as follow: fumaric acid is suspended in water and calcium base is added. The resulting mixture is heated up to 70˜90° C. and kept under stirring for 2˜8 hours, and then concentrated under reduced pressure. The resulting dried substance is added into ethanol, and the mixture is vigorously stirred. The inner salt of L-carnitine is added, and the mixture is reacted for 1-6 hours at 60˜70° C. and then cooled for 2˜8 hours. L-carnitine calcium fumarate is obtained by filtration.

### TECHNICAL FIELD

The invention relates to stable, non-hygroscopic and pharmacologically acceptable salts of L-carnitine, more particularly, to L-carnitine calcium fumarate and its preparation method and application. This method is involved with the producing of food additives and pharmaceutical intermediates which may serve as constituent of nutritional supplements for human body or animal feed supplements, including compositions of nutritional supplement and calcium replenishment.

### BACKGROUND ART

The efficacy of L-carnitine has been extensively reported. L-carnitine is an important enrichment element of food nutrition, finding wide use in food, for example, in infantile powdered milk, slimming food, nutritional supplements for athletes and the aged and in animal feed. Besides, L-carnitine has obvious therapeutic efficacy. It has been proved that L-carnitine and its product series have therapeutic effects on cardiovascular, liver, kidney and neuromuscular diseases, hyperlipidemia, diabetes, etc. It is also reported that L-carnitine may enhance reproductive capacity.

It is already known that pharmacologically acceptable salts of L-carnitine have the same therapeutical/nutritional activities as the inner salts, without any toxic or side effects. These pharmacologically acceptable salts can improve the stability and hygroscopicity of the inner salts and have been put to wide practice, including L-carnitine acid fumarate (U.S. Pat. No. 4,602,039, Sigma-Tau) and L-carnitine L-tartrate (U.S. Pat. No. 5,073,376, Lonza) which are very common on the market at present and also the more recently L-carnitine galactarate (U.S. Pat. No. 5,952,379, Sigma-Tau).

However, L-carnitine tartrate is still of higher hygroscopicity, and will deliquesce at the relative humidity slightly exceeding 60% and the anion part of tartaric acid itself does not have any nutritional or therapeutic efficacy. L-carnitine calcium fumarate according to our invention is non-hygroscopic and can withstand greater relative humidity than L-carnitine L-tartrate. Moreover, fumaric acid itself is a key intermediate of tricarboxylic acid cycle in metabolism of organisms and will, once taken in, quickly join metabolism process to play the role as a substance of energy.

Trace metal elements such as magnesium, calcium and so on are essential to human body, and therefore, they are extensively needed as nutrients. Epidemiological studies have revealed that there is a distinct correlation between the incidence of cardiac ischaemia and the calcium/magnesium ratio in the diet and drinking water. For a detailed review of the physiological and pharmacological activities and therapeutic uses of magnesium and calcium, reference is made to Goodman and Gilman's “The pharmacological basis of therapeutics” (1990). Moreover, disorders of calcium and magnesium concentration are dealt with in Current Medical Diagnosis & Treatment (1999).

Combination of L-carnitine with these metal ions will produce well water-soluble salts to be easily absorbed by human body so that the nutritional and therapeutic efficacy of L-carnitine may be enhanced. Several such salts have been disclosed, but they are mainly focused on salts containing magnesium, such as L-carnitine magnesium fumarate and lower alkanoyl-L-carnitine magnesium fumarate (U.S. Pat. No. 6,051,608, Sigma-Tau); L-carnitine magnesium tartrate and lower alkanoyl-L-carnitine magnesium tartrate (WO 98/45250, Sigma-Tau); L-carnitine magnesium citrate (U.S. Pat. No. 5,071,874, Lonza), and lower alkanoyl-L-carnitine magnesium citrate (WO 98/44918, Sigma-Tau). For salts combined with calcium ions, until now, only L-carnitine calcium galactarate has been disclosed (WO 02059075). But, it has not been stated about L-carnitine calcium fumarate in any document.

### DISCLOSURE OF THE INVENTION

An object of present invention is to overcome the present shortcomings of aforementioned technologies, and provides new pharmacologically acceptable salts of L-carnitine, namely L-carnitine calcium fumarate and also a preparation method and applications thereof. Having good water solubility as well as better and more nutritional and therapeutical activities than the corresponding inner salts, the L-carnitine calcium fumarate has the advantages of being non-hygroscopic, stable in chemical property, and suitable for oral administration.

The technical solution proposed in present invention is as follows: the formula of the L-carnitine calcium fumarate is:

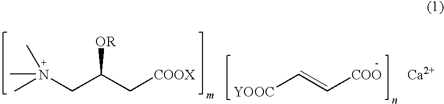

Wherein R=H, or a straight or lower branched chain alkanoyl containing 2-6 carbon atoms;
m=1 or 2;
n=1 or 2; and
when m=1 and COOX=COO⁻, n=1, and COOY=COO⁻ or n=2, and COOY=COOH;
when m=2 and COOX=COOH, n=2 and COOY=COO⁻;
when m=2, and COOX=COO⁻, n=1, and COOY=COO⁻.
   Particularly preferable structure contains:

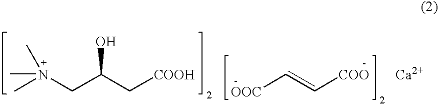

And also

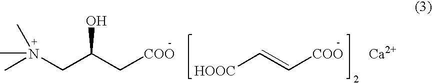

Besides the important role played by calcium on bones and teeth, L-carnitine calcium fumarate according to the present invention has functions of maintaining cell survival and contributing to nerve conduction, immune system maintenance, blood coagulation, metabolism, muscle contraction and heart cell nourishing. Being able to promote activities of many enzymes and catalyze thrombin in body, calcium is essential to the maintenance of acid base balance and to the maintenance and regulation of many biochemical processes in body. Lack of calcium may easily lead to neoplastic hyperplasia of cartilage, osteoporosis, rickets, sciatica, dental caries, and hoary hair, cause easily muscle cramp, degradation of myocardial function, heart disease, degradation of reproductive capacity, dysmenorrheal as well as hypertarachia, mental disorders, memory decline, fatigability and allergic reaction, and increase pathogenicity rates of colorectal cancer, hypertension, bone deformities and spasticity. Therefore, as the source of vitality, calcium is an indispensable element to human body and also a common constituent in a variety of nutritional supplements. However, majority of natural calcium and synthetic calcium formulations are slightly soluble in water, and thus their absorbability are not satisfying. Before being absorbed, they have to be activated first by gastric acid in stomach and turned into active calcium, namely, ionic calcium. Therefore, it is of great significance to change calcium to water-soluble salt, namely, active calcium which may be, potentially, used as food additives or nutritional supplements and absorbed sufficiently.

Ingestion of large amount of calcium lactate may cause fatigue and excessive calcium gluconate and calcium chloride are harmful to diabetics. L-carnitine calcium fumarate as proposed have no side effects as mentioned above and are suitable to be used, with greater nutritional efficacy, to substitute those existing calcium supplements.

Preparation of the proposed L-carnitine calcium fumarate is as follows:

Fumaric acid is suspended in water, calcium base is added, and the resulting mixture is heated to the temperature of 70˜90° C. with violently stirring till it turns to be complete solubilization. Allowed to react for 28 hours, then the solution is concentrated under reduced pressure. The resulting dried calcium fumarate is added into ethanol or methanol and the mixture is stirred vigorously. The inner salt of L-carnitine is added, and then allowed to react for 1˜6 hours at 60˜70° C. Thereafter, the resulting substance is cooled at −5˜5° C. for 2˜8 hours. L-carnitine calcium fumarate is obtained by filtration. The final product is turned out after dying process. This produced salt has good fluidity, non-hygroscopicity and better water-solubility.

In the embodiments given in present invention, the said calcium base is Ca(OH)₂, CaO or CaCO₃. Since fumaric acid and inorganic calcium base are poor in water solubility, the amount of solvent water to be used is 80˜140 mL for every gram of calcium base when calcium fumarate is prepared.

In the embodiments of present invention as given above, water is used as the solvent and the objective product is hard to be precipitated from the reaction of calcium fumarate with L-carnitine. Therefore, ethanol or methanol rather than water is selected as the solvent.

The scope of present invention is further related to application of such L-carnitine calcium fumarate. Namely, it may be used, in form of compositions, as a dietary or nutritional supplement for human body or as a nutritional supplement for animals, wherein said compositions contain, as an active ingredient, one of any said L-carnitine calcium fumarate in structural formula 1-3, including above-mentioned L-carnitine calcium fumarate compositions which are orally administrable, non-hygroscopic and pharmacologically acceptable, including compositions which are suitable to be taken orally, non-hygroscopic and stable; and also including one or more pharmacologically acceptable excipients in which the compositions may be optionally contained, or one or more active ingredients with which are well-known to the experts in pharmacy and food technology.

Particularly preferred is solid or liquid, orally administrable such as tablet, capsule, granule, powder, oral liquid and so on. The compositions may comprise one form of any L-carnitine calcium fumarate shown as structure formula (1). The amount of inner salt of L-carnitine or alkanoyl L-carnitine for a single unit dose is 50-2000 mg, preferably, 100-1000 mg.

For instance, a composition for preparing tablets is following:

| | | |
|---|---|---|
| L-carnitine calcium fumarate: | 500 | mg |
| Starch: | 20 | mg |
| Talc: | 5 | mg |
| Microcrystalline cellulose: | 30 | mg |
| Magnesium stearate: | 2 | mg |
| | 557 | mg |

The proposed composition can be used as dietary supplements for human body or as feed supplements for animals, or as a kind of supplement for calcium supplying.

Compared with the prior arts, the beneficial effects of present invention are as follows: the proposed L-carnitine calcium fumarate is suitable to be taken orally, stable in chemical property, stolid to moisture, convenient in storage and transportation; and what is more important, it is ready to be used for making solid preparations. In addition, compared with the other inner salts, it will have stronger and more functions of nutrition and treatment, especially increasing the efficiency of calcium replenishment.

### EMBODIMENTS

Following non-limiting examples are intended to further describe such a stable, non-hygroscopic L-carnitine calcium fumarate according to present invention including its preparation and applications.

### Example 1

### Preparation of L-Carnitine Calcium Fumarate (2:2:1)

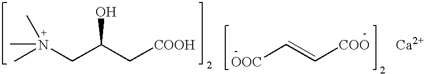

Fumaric acid (11.6 g, 0.1 mol) is suspended in 200 mL of water and mixed by stirring. Finely powdered calcium hydroxide (totally 3.7 g, 0.05 mol) is added to the mixture for two times at an interval of half an hour. The resultant mixture is heated up to the temperature of 70˜90° C. and then stirred violently till it is complete solubilization. Allowed to react for two hours, then the solution is concentrated under reduced pressure. L-carnitine (16.12 g, 0.1 mol) is suspended in 200 mL of ethanol and allowed to react for 3 h at 60˜70° C. After falling down to room temperature, the mixture is cooled at −5˜5° C. for two hours. The product of L-carnitine calcium fumarate is finally obtained by filtration, and in this case 28 g of product can be obtained after drying, the yield being 95%.

The obtained L-carnitine calcium fumarate is in uniform powder form with good fluidity, more than 98% of which is smaller than 40 mesh size. This product has good water-solubility with a solubility of 1.8 g/100 mL water. There will be no caking or sticking phenomena occurring at 25° C. and relative humidity of 60±5% and after 24 hours exposure to air. The product has a calcium content of 6.76% with good non-hygrosopicity.

DSC: decompose at 160° C., with not melt.
[α]_{D}²⁰ = 11.37(c = 1%, H₂O)
pH: 3.98
Elementary analysis for C₂₂H₃₆N₂O₁₆Ca

| | | | | |
|---|---|---|---|---|
| | C % | N % | H % | Ca % |
| Calculated (with 7.5% water): | 44.5 | 4.7 | 6.1 | 6.7 |
| Found: | 41.8 | 4.3 | 6.33 | 6.7 |
| HPLC: | | | | |

| | |
|---|---|
| Column: | APS-2 HYPERSIL (NH₂) (5 μm) |
| | 250 × 4.6 mm |
| Temperature: | 30° C. |
| Mobile phase (v/v): | KH₂PO₄/acetonitrile (70/30)(V/V) |
| pH: | 4.0 with H₃PO₄ |
| Detection wavelength(λ): | 205 nm |
| Flow rate: | 1.0 ml/min |
| Carnitine: | Rt = 5.3 min |
| Fumaric acid: | Rt = 12.2 min |

### Example 2

### Preparation of L-Carnitine Calcium Fumarate (1:2:1)

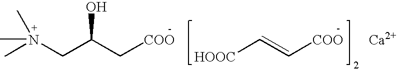

Fumaric acid (23.2 g, 0.2 mol) is suspended in 740 mL water and calcium hydroxide (7.4 g, 0.1 mol) is added under stirring. The mixture is heated up to 70° C. under stirring violently till it is complete solubilization and allowed to react for two hours, then concentrated under reduced pressure. The dried substance is suspended in 200 mL ethanol under stirring thoroughly, then L-carnitine (16.12 g, 0.1 mol) is added and allowed to react for 3 h at 60˜70° C. Thereafter the obtained substance is cooled at −5˜5° C. for five hours. L-carnitine calcium fumarate is thus obtained by filtration, and in this case 39.5 g of product can be obtained after drying, the yield being 95%. The water-solubility of this powder product smaller than 40 mesh size is 3.5 g/100 mL (cold water) and has a calcium content of 9.28%.

The obtained L-carnitine calcium fumarate is in uniform powder form and has good fluidity, greater than 98% of which is smaller than 40 mesh in granular size. Its water-solubility is good and the solubility is 3.5 g/100 mL water. There will be no caking or sticking phenomena occurring at 25° C. and relative humidity of 60±5% and after 24 hours exposure to air. The product has a calcium content of 9.28%, with better non-hygrosopicity.

DSC: decompose at 136° C., with not melt.
[α]_{D}²⁰ = −10.2 (c = 1%, H₂O)
pH: 3.82
Elementary analysis for C₁₅H₂₁NO₁₁Ca

| | | | | |
|---|---|---|---|---|
| | C % | N % | H % | Ca % |
| Calculated (with 7.5% water): | 38.62 | 3.00 | 5.36 | 9.28 |
| Found: | 37.06 | 3.10 | 5.13 | 9.22 |
| HPLC: | | | | |

| | |
|---|---|
| Column: | Bonchrom-C18(5 μm) 250 × 4.6 mm |
| Temperature: | 30° C. |
| Mobile phase: | 0.05MKH₂PO₄/acetonitrile(60/40)(v/v) |
| pH: | 3.0 with H₃PO₄ |
| Detection wavelength(λ): | 205 nm |
| Flow rate:: | 0.6 ml/min |
| L-carnitine: | Rt = 5.6 min |
| Fumaric acid: | Rt = 12.3 min |

This invention adopts the form of a kind of composition to be used as a dietary or nutritional supplement for human or as a nutritional supplement for animals, wherein said composition comprise one of any L-carnitine calcium fumarate given in structure formula I-3 as an active ingredient, pharmacological acceptable excipients in any form required may be made out of this composition, for example, oral solid preparations such as tablets, capsules, masticable tablets, or liquid preparations such as oral liquid. The composition comprises one form of L-carnitine calcium fumarate shown in structure formula I-3 and the amount of L-carnitine inner salt or alkanoyl L-carnitine inner salt for a single unit dose being 50-2000 mg, preferably, 100-1000 mg wherein constituents of one tablet preparation are as follows:

| | | |
|---|---|---|
| L-carnitine calcium fumarate: | 500 | mg |
| Starch: | 20 | mg |
| Talc: | 5 | mg |
| Macrocrystalline cellulose: | 30 | mg |
| Magnesium stearate: | 2 | mg |
| | 557 | mg |

1. A L-carnitine calcium fumarate, wherein the chemical structure thereof is 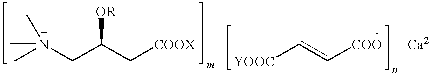
wherein R=H, or R equals to a straight or branched lower alkanoyl containing 2-6 carbon atoms,
m=1 or 2,
n=1 or 2,
wherein when m=1 and COOX=COO⁻, n=1, COOY=COO⁻ or n=2, and COOY=COOH,
wherein when m=2 and COOX=COOH, n=2 and COOY=COO⁻,
wherein when m=2, and COOX=COO⁻, n=1, and COOY=COO⁻.

2. The L-carnitine calcium fumarate, as recited in claim 1, wherein the chemical structure thereof is

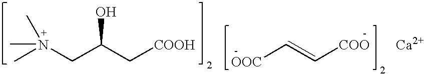

3. The L-carnitine calcium fumarate, as recited in claim 1, wherein the chemical structure thereof is

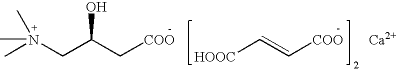

4. A method of preparing a L-carnitine calcium fumarate, comprising steps of:
(a) suspending fumaric acid in water;
(b) adding calcium base;
(c) heating said resulting mixture is heated up to 70˜90° C.;
(d) stirring said mixture till complete solution is achieved;
(e) allowing said mixture to react for 2˜8 hours;
(f) concentrating said solution is under reduced pressure;
(g) adding said dried calcium fumarate into ethanol or methanol and stirring vigorously;
(h) adding inner salt of L-carnitine;
(i) allowing said mixture to react for 1˜6 hours at 60˜70° C.;
(j) cooling at −5˜5° C. for 2˜8 hours; and
(k) obtaining said L-carnitine calcium fumarate by filtration.

5. The method, as recited in claim 4, wherein said calcium base is selected from the group consisting of Ca (OH)₂, CaO, or CaCO₃.

6. The method, as recited in claim 4, wherein the amount of solvent water as used for every gram of calcium base is 80˜140 mL.

7. A composition used as a dietary or nutritional supplement for human or as nutritional supplement for animals, comprising a L-carnitine calcium fumarate as recited in claims 1 as an active ingredient.

8. The composition, as recited in claim 7, wherein said composition is in the form of powder, granule, tablet, capsule, or oral liquid which is used in solid or liquid oral administrable preparations.

9. The composition, as recited in claim 7, wherein the unit dose content of said L-carnitine calcium fumarate is 100-1000 mg therein.

10. The composition, as recited in claim 8, wherein the unit dose content of said L-carnitine calcium fumarate is 100-1000 mg therein.

11. A composition used as a dietary or nutritional supplement for human or as nutritional supplement for animals, comprising a L-carnitine calcium fumarate as recited in claims 2 as an active ingredient.

12. The composition, as recited in claim 11, wherein said compositions is in the form of powder, granule, tablet, capsule, or oral liquid which is used in solid or liquid oral administrable preparations.

13. The composition, as recited in claim 11, wherein the unit dose content of said L-carnitine calcium fumarate is 100-1000 mg therein.

14. The composition, as recited in claim 12, wherein the unit dose content of said L-carnitine calcium fumarate is 100-1000 mg therein.

15. A composition used as a dietary or nutritional supplement for human or as nutritional supplement for animals, comprising a L-carnitine calcium fumarate as recited in claims 3 as an active ingredient.

16. The composition, as recited in claim 15, wherein said compositions is in the form of powder, granule, tablet, capsule, or oral liquid which is used in solid or liquid oral administrable preparations.

17. The composition, as recited in claim 16, wherein the unit dose content of said L-carnitine calcium fumarate is 100-1000 mg therein.

18. The composition, as recited in claim 17, wherein the unit dose content of said L-carnitine calcium fumarate is 100-1000 mg therein.
